(19) United States
(12) Patent Application Publication
(10) Pub. No.: US 2005/0033184 A1
(43) Pub. Date: Feb. 10, 2005

(54) Arrangement and method for measuring shape of basically two dimensional objects

(76) Inventor: Ralf Christoph, Giessen (DE)

Correspondence Address: DENNISON, SCHULTZ, DOUGHERTY & MACDONALD, 1727 KING STREET, SUITE 105, ALEXANDRIA, VA, 22314, US

(21) Appl. No.: 10/499,816

(22) PCT Filed: Mar. 14, 2003
(86) PCT No.: PCT/EP03/02719

(30) Foreign Application Priority Data
Mar. 14, 2002 (DE) 102 11 760.8

(51) Int. Cl. A61B 6/00

(52) U.S. Cl. 600/476

(57) ABSTRACT

A device and method for measuring geometries or structures of essentially two-dimensional objects involving the use of an image processing sensory mechanism. According to the invention, the object to be measured is placed on an object supporting surface and is measured on the side of the object supporting surface by means of an image processing sensory mechanism that can be displaced underneath the object in a plane running parallel to the object supporting surface.

The invention concerns an arrangement for measuring the shapes or structures of basically two dimensional objects using an image processing sensor system whereby the object to be measured is arranged on an object-bearing surface and the image processing sensor system, such as a CCD camera, is arranged on one side of the object. Furthermore, the invention relates to a method for measuring shapes or structures of a basically two dimensional object using an image processing sensor system, whereby the object to be measured is arranged on an object-bearing surface and the image processing sensor system, such as a CCD camera, is arranged on one side of the object.

Preferably coordinates measuring devices with an image processing sensor system, such as CCD cameras, are used for measuring the shape of chiefly two dimensional objects such as workpieces or tools, especially for the mechanical recording of quality features. These devices are generally constructed in such a way that the object to be measured is illuminated from below, moved using a mechanical stage and measured object structures of interest are measured in an image processing sensor. The disadvantage of the construction principle described consists in that the image processing sensor must be refocused when objects have different thicknesses. The refocusing likewise necessary on the object when the smallest height gradations occur delays the measuring sequence.

It is furthermore usual to position the image processing sensor system for measuring certain features at the respective location of the feature, then record and later calculate the workpiece contours. A general overview of the measured object is consequently not obtained.

Furthermore, individual regions of the measured object are brought up several times in order to record features lying close to one another. This, likewise, leads to lengthening the measuring time.

So-called scanner systems are also known where greater sections of a region can be scanned with line-like sensors. The disadvantage of such systems lies in that the image information from a linear scanning motion in one direction and the sensor shape in a second direction are joined together. The particular shape of the sensor system likewise requires imaging optical systems that basically do not permit a high grade imaging. In the end, the measuring devices manufactured according to this principle are outfitted with only a low exactitude.

The present invention is based on the problem of developing further an arrangement and a method of the type mentioned at the beginning in such a way that two-dimensional objects or their shapes, especially object edges and corners and margins, can be measured very rapidly with high accuracy.

To solve the problem, it is basically provided in accordance with the arrangement that the image processing sensor system is located beneath the object and can be adjusted on a plane running parallel to the object-bearing surface.

In particular, the mobile image processing sensor system is arranged in a closed housing that is closed off on the object side by a transparent surface on which the object can be positioned. Obviously there also exists the possibility of arranging the object on a separate object table at a distance from the transparent surface.

In a further development, the invention provides that an illumination apparatus is arranged above the object or workpiece bearing surface, preferably as a luminous surface.

It is in particular provided that the arrangement includes housing with a closed lower part and a roof. The closed lower part includes the sensor unit with optical unit as well as a drive and is provided with a transparent covering, such as a glass plate on which the object to be measured can be positioned. The cover itself has the illumination apparatus, whereby a measurement can only take place when the cover masks the lower part of the housing, thus closing it off Consequently, the object is surrounded on the peripheral side completely by the housing during measuring, thus by the housing and the cover, so that an unintended dislocation of the object or other change influencing the measurement cannot take place.

Additionally, it can be provided that the object is surrounded by light sources beaming in the direction of the object, such as light diodes, in order to illuminate the object on the sensor side.

A telecentric objective with a great depth of field can be used as an imaging optical system for the image processing sensor unit. The depth of field can, for example, come to 50 mm without a restriction taking place through this.

The position of the image processing sensor unit can be adjusted with this allocated XY drive, whereby the position is measurable through corresponding scale systems.

An image memory can be connected as an image processing sensor that represents the size of a desired, especially overall measuring range of the device. Furthermore, an evaluation computer unit can be allocated to the image memory for the measuring range, especially overall measuring range, that undertakes the geometric evaluation on the overall image content.

Preferably the image processing sensor system includes matrix-like image processing sensors and is constructed as a CCD matrix camera.

A method for measuring shapes of basically two-dimensional objects of the type mentioned at the beginning is distinguished in that the image processing sensor system is arranged adjustably on a plane beneath the object-bearing surface that runs parallel to the object-bearing surface.

Accordingly, an image processing sensor with an upward direction of view is arranged mobile on a plane beneath the measured object.

Moreover, images can be recorded on several positions of the measuring region using the image processing sensor system and these can be compiled into an overall image by computer in the image memory. There also exists the possibility of recording images separately over the entire measuring region and joining these together into an overall image. Moreover, the overall image can be evaluated with respect to geometric features with an image processing system. For example, the field of vision of the sensor can come to 50×80 mm² and the measuring region to 400×200 mm to name some figures only by way of example.

The theory of the invention makes it possible to reduce or avoid the disadvantages immanent in the state of the art. This takes place in accordance with the invention in that the moved image processing sensor system is arranged beneath the object and for example beneath a glass place with a direction of view on the object, hence upward. This leads to the fact that the measuring object regions and edges of the measured objects can respectively come to lie on the same plane independently of thickness. Focusing the sensor unit is consequently not necessary.

Furthermore, the inventive use of an optical system with sufficient depth of field makes possible measuring even graduated objects in one place without a focusing process.

To optimize measuring time, the entire measuring field or sections of the measuring field can be selectively scanned by lining up positions of the image processing sensor unit. An overall image is virtually generated in the adjoining image processing computer. The metrological evaluation takes place in the overall image in a single operation. Consequently, positioning procedures are spared and a general overview over the object to be measured is obtained.

Even sections of the measuring field can be represented as a partial overall image and then evaluated in an image processing system.

The disadvantages of the state of the art are in particular avoided by the exact positioning of matrix-like image processing sensors.

It is preferably provided that an optical system with variable work distance is used for recording the object or regions of the latter. Nonetheless, in particular an optical system that has a zoom optical system can be used that contains at least two lens groups that are respectively axially displaceable separately by a motor. Refer to this extent to WO 99/53268 to the disclosure of which reference is explicitly made.

It is proposed in a refinement of the invention that first a crude aligning of the image processing sensor on the position of the object or part of the object to be measured takes place, whereby when aligning the image processing sensor, the latter is moved with an acceleration a₁>0 mm/s², in order then to brake the image processing sensor and to measure the position when the image processing sensor is moved at an acceleration a₂ with 0 mm s₂≦a₂<a₁. If need be, the object can moreover additionally be energized with a light flash, or a CCD camera with shutter can be used as the image processing center. A correlation between the motion of the sensor and the image to be respectively recorded takes place through measures in this regard, whereby an apparent stoppage of the image processing sensor is realized through the light flash or the shutter with the consequence that measurements are conducted such as if the image processing sensor would stand still during the measurement.

Further details, advantages and features of the invention emerge not only from the claims, the features to be inferred from these-by themselves and/or in combination, but also on the basis of the following description of preferred designs to be inferred from the drawing, wherein:

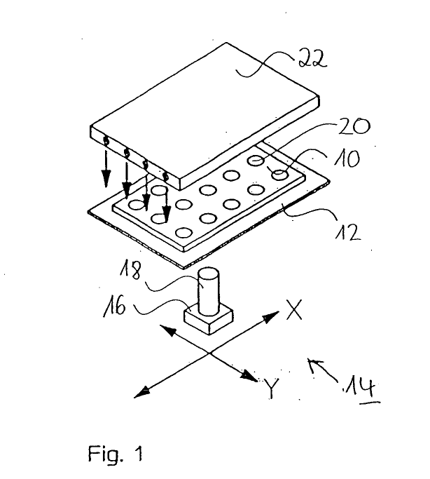

FIG. 1 illustrates a basic representation of an arrangement for measuring a two-dimensional object,

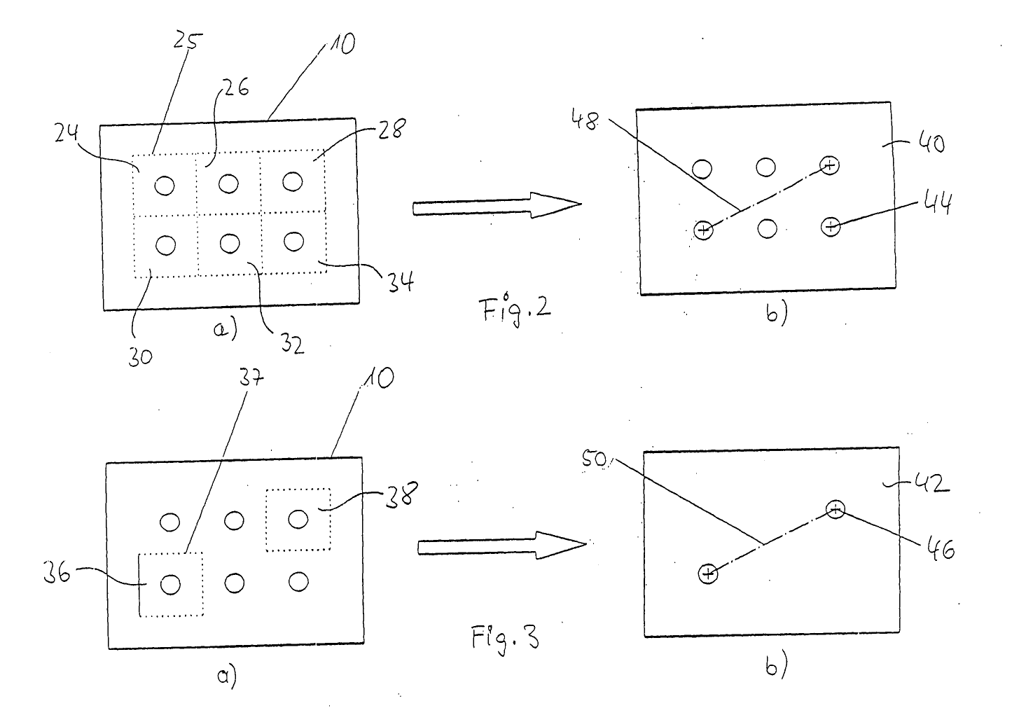

FIG. 2a +2b illustrates a basic representation of a first design of a measurement method and

FIG. 3a +3b illustrates a basic representation of a second design of a measurement method.

In FIG. 1, an arrangement for measuring an essentially two dimensional object 10 is very basically that is arranged on an object-bearing surface 12. This is constructed transparently and in particular as a glass plate in accordance with the invention in order to be able to measure the object 10 from underneath. An image processing sensor system 14 is arranged adjustable in the X and Y direction of a coordinates measuring device beneath the object-bearing surface. The image processing sensor system consists preferably of a CCD matrix camera 16 in front of which an optical system 18 is arranged, especially in the form of a telecentric objective.

Moreover, the object-bearing surface 12 can be the surface of a housing in which the image processing sensor system 14 can be adjusted in the X and Y direction in relation to the object 10. The housing surface is moreover transparent, whereby the object 10 either lies directly on the housing surface or at an equidistant spacing toward the latter.

Due to the fact that the moved image processing sensor system 14 is arranged beneath the object 10 and beneath the object-bearing surface 12 particularly constructed as a glass plate with a direction of view to the object 10, a focusing of the sensor system 16 is no longer necessary with a sufficient depth of field of the object 18 since the object regions to be measured come to lie in the same plane as edges of boreholes 20 in the design independently of the thickness of the object 10, namely on the object-bearing surface 12 which has a constant distance independent from the position of the image processing sensor system 14 in relation to the latter.

If the objective 10 is arranged directly upon a glass plate in the design, then obviously there also exists the possibility of arranging the object 10 spaced toward this, but at an equidistant spacing.

An illumination, especially in the form of a flat light field 22, is provided for illuminating the object above this, thus on the side of the object 10 lying opposite the image processing sensor system 14.

The flat light field should moreover be incorporated into a cover that closes the housing in which the image processing sensor system 14 is arranged with the optical system 18 and the drive and which is closed off by a transparent element such as a glass plate in reference to the field of illumination side on which the object to be measured can be positioned. Moreover, usually a measurement should only be conducted when the cover containing the light field 22 completely covers the housing, thus closes it on the glass plate side.

In order to conduct precise measurements with great speed, it is provided in accordance with the invention that the image processing sensor system 14 records images at several positions 24, 26, 28, 30, 32, 34, 36, 38 of the object 10 to be measured, which basically correspond to the respective field of sight 25, 37 in FIGS. 2a and 3a in the image processing sensor system, and which are represented by squares framed by dotted lines, in order then to join the respective images into an overall image according to FIG. 3b by computer in an image memory in accordance with FIG. 2b, or in the case of imaged recorded separately (FIG. 3a). Geometric features such as position of a measuring site 44, 46 or distance 48, 50 of measuring points or measuring sites can the be evaluated on the basis of the respective overall image 40 or 42. The field of vision 24, 25, 26, 28, 30, 32, 34, 36, 37, 38 can, for example, amount to a size of 50×80 mm² and the measuring region to 400×200 mm without this being understood as restrictive.

In other words, the entire field of measurement (FIG. 2a) or sections of the field of measurement (FIG. 3a) can be selectively screened by lining up positions of the image processing sensor such as the matrix CCD camera 16 for optimizing measurement time in accordance with the invention. On the basis of this, an overall image 40, 42 is generated virtually in a connected image processing computer, whereby the metrological evaluation takes place in the overall image in a single operation. Consequently positioning processes are spared and an overall overview on the object 10 to be measured is obtained. The disadvantages of the state of the art are avoided through these measures by exact positioning of matrix-like image processing sensors.

Independently of this, the measurement process can be optimized in that first of all a crude aligning of the image processing sensor on the object to be measured takes place, whereby the image processing sensor is moved with an acceleration a₂>0 mm/s² when aligning the image processing sensor and the position is measured when the image processing sensor is moved at an acceleration a₂ with 0 mm/s²≦a₂<a₁. Moreover, an image processing sensor in the form of a CCD camera with shutter can be used, due to which the advantage results that an apparent stoppage of the image takes place during measurement regardless of the motion of the sensor. The same can be realized with a light flash.

In other words, the image processing sensor only moves crudely on the position to be measured and is then measured when the image processing sensor is moved further, but basically not accelerated. For measurement, it can be moved with a speed of v₁ of, for example 50 to 200 mm/s. Moreover the image storage necessary for measuring can be recognized in the image processing sensor by reaching a target area. Braking can thus be introduced by optical recording of the area of the object containing the position using the image processing sensor.

Moreover, a motion of the image processing sensor can take place in such a way that at a speed of vi, the object or measuring region or measuring points of the latter are measured, and the image processing sensor is subsequently strongly accelerated, for example, to a value of ca. 5000 to 15,000 mm/s in order then to be crudely aligned at an acceleration of 0 mm/s² at a speed v₂ between 400 and 600 mm/s on the measuring region or measuring point. Then a braking of the image processing sensor to speed v₁ takes place in the range preferably between 50 mm/s and 150 mm/s in order to perform measurements. During this time the object or the region to be measured can be activated by light flashes or the shutter of the image processing sensor can be opened and closed at the desired frequency. After measurement has taken place, the image processing sensor is then accelerated in the direction previously described in order to be aligned on a new measuring point or region.

1. Arrangement for measuring shapes or structures of basically two dimensional objects (10) by means of an image processing sensor system (14), whereby the object to be measured is arranged on an object-bearing surface (12) and the image processing sensor, such as a CCD camera (16), is arranged on one side of the objects, wherein the image processing sensor system (14) is arranged beneath the object (10) and is adjustable on a plane running parallel to the object-bearing surface (12).

2. Arrangement according to claim 1, wherein the image processing sensor system (14) includes matrix-like image processing sensors, especially a CCD matrix camera (16).

3. Arrangement according to claim 1, wherein the image processing sensor system that is movable in the X and Y direction of a coordinates measuring device is arranged in a closed housing that is transparent or translucent on the object side and the object-bearing surface (12) is or runs parallel to the object-bearing surface.

4. Arrangement according to claim 1, wherein an illumination apparatus (22), preferably in the form of a luminous surface, is arranged above the object to be measured or the object-bearing surface (12).

5. Arrangement according to claim 4, wherein the illumination apparatus (22) is incorporated into a cover closing the housing on the object-bearing surface side.

6. Arrangement according to claim 1, wherein the imaging optical system (18) of the image processing sensory system (14) is a telecentric objective with great depth of field.

7. Arrangement according to claim 6, wherein the imaging optical system (18) is surrounded, preferably concentrically, by illumination elements such as light diodes for illumination of the image processing sensor side surface of the object (10).

8. Arrangement according to claim 1, wherein the position of the image processing sensor system (16) is adjustable with this allocated x-y drive and the position is measurable through corresponding scale systems.

9. Arrangement according to claim 1, wherein an image memory is connected to the image processing sensor (16) that represents the magnitude of a desired measuring region (40, 42), especially the overall measuring region of the arrangement.

10. Arrangement according to claim 9, wherein an evaluation computer unit is allocated to the image memory (40, 42) for the measurement region which undertakes the geometrical evaluation on the overall image content.

11. Method for measuring shapes or structures of a basically two dimensional object using an image processing sensor system (14) whereby the object to be measured is arranged on an object-bearing surface (12) and the N image processing sensor system such as a CD camera (16) is arranged on one side of the object, wherein the image processing sensor (14) is arranged beneath the object-bearing surface (12) and is adjustable on a plane which runs parallel to the object-bearing surface.

12. Method according to claim 11, wherein the image processing sensor system (14) moves in the X and Y direction of a coordinates system and is arranged in a stationary closed housing that is closed off on the object side by a transparent surface on which the object (10) is laid or toward which the object is arranged at a distance on a plane running parallel to the surface.

13. Method according to claim 11, wherein an illumination apparatus (22), preferably in the form of a luminous surface, is arranged above the object-bearing surface (12) or the object to be measured (10).

14. Method according to claim 13, wherein the illumination apparatus (22) is incorporated into a cover through which the housing can be masked, whereby a measurement of the object (10) is prevented when the cover exposes the object-bearing surface (12).

15. Method according to claim 11, wherein a telecentric objective with great depth of field is used as an imaging optical system (18) of the image processing sensor (16).

16. Method according to claim 11, wherein the position of the image processing sensor (16) is adjusted with this allocated x-y drive and the position is measured by appropriate scale systems.

17. Method according to claim 11, wherein an image memory is connected to the image processing sensor (14) that represents the magnitude of a desired or overall measuring region (40, 42) of the arrangement.

18. Method according to claim 17, wherein an evaluation computer unit is allocated to the image memory for the desired or overall measuring region (40, 42) which undertakes the geometric evaluation on in particular the entire image content.

19. Method according to claim 11, wherein images are recorded at several positions (24, 26, 28, 30, 32, 34, 36, 38) using the image processing sensor system (14) and these are assembled by computer in the image memory into an overall image (40, 42).

20. Method according to claim 11, wherein images (24, 26, 28, 30, 32, 34, 36, 38) are recorded that are distributed over the entire measuring region and they are joined together into an overall measurement image (40, 42).

21. Method according to claim 11, wherein the overall image (40, 42) is evaluated with respect to geometrical features with an image processing system.

22. Method according to claim 11, wherein the following operations are conducted for measuring the object (10) or a region thereof:
crude aligning of the image processing sensor (14) on positions of the object (10) to be measured, whereby when aligning the image processing sensor, this is moved with an acceleration a₁>0 mm/s², and
braking the image processing sensor in measuring position when the image processing sensor is moved at an acceleration a₂ with 0 mm/s²≦a₂<a₁.

23. Method according to claim 22, wherein the object (10) is acted upon with a light flash during measurement or a CCD camera with shutter is used as an image processing sensor (16).

24. Method according to claim 15, wherein an imaging optical system with variable working distance, especially an imaging optical system with a zoom optical system, is used that contains at least two respective lens groups axially and separately displaceable by a motor.
